(19) United States
(12) Patent Application Publication
(10) Pub. No.: US 2021/0181185 A1
(43) Pub. Date: Jun. 17, 2021

(54) DENDRITIC CELL RECRUITMENT FROM BLOOD TO BRAIN IN NEURODEGENERATIVE DISEASE

(71) Applicant: MindImmune Therapeutics, Inc., Kingston, RI (US)

(72) Inventors: Brian Michael Campbell, Quincy, MA (US); Frank Samuel Menniti, Mystic, CT (US); Robert Brelsford Nelson, Gales Ferry, CT (US); Stevin Howard Zorn, Salem, CT (US)

(73) Assignee: MindImmune Therapeutics, Inc., Kingston, RI (US)

(21) Appl. No.: 16/761,350

(22) PCT Filed: Nov. 8, 2018
(86) PCT No.: PCT/US2018/059761

§ 371 Date: May 4, 2020

Related U.S. Application Data
(60) Provisional application No. 62/583,959, filed on Nov. 9, 2017.

(51) Int. Cl. G01N 33/50

(52) U.S. Cl. CPC G01N 33/5047; G01N 2800/2814; G01N 2800/2821; G01N 33/5091

(57) ABSTRACT

The present disclosure provides methods of treating neurodegenerative diseases, including administering agents that block dendritic cells from entering the brain from the blood. The present further describes methods for detecting the selective migration of dendritic cells from blood into brain as a marker of human neurodegenerative disease. The present disclosure further describes methods that can be used to evaluate and compare different embodiments of methods detecting migration of dendritic cells using transgenic mice that overexpress various mutations associated with induction of distinct neurodegenerative disease pathologies. These present disclosures are particularly useful in the design and evaluation of clinical trials for neurodegenerative diseases, such as Alzheimer's disease, small vessel diseases that include cerebral amyloid angiopathy, and fronto-temporal dementias, and specifically for the design and evaluation of clinical trials of agents that block dendritic cells from entering the brain for therapeutic purpose.

### FIELD

The present disclosure relates to novel methods and assays for identifying when migration of dendritic cells from blood into brain is occurring in animals and humans. The present disclosure further relates to methods of treating neurodegenerative disease through the administration of compounds demonstrated through the methods of this patent to decrease the migration of dendritic cell precursors from blood into brain. These methods are particularly useful in the design and evaluation of clinical trials for neurodegenerative diseases, such as Alzheimer's disease, small vessel diseases that include cerebral amyloid angiopathy, and fronto-temporal dementias. The methods and assays of the present disclosure are particularly useful for identifying and stratifying individuals for potential inclusion or exclusion in clinical trials, diagnosing and staging neurodegenerative disease progression in individual patients (or as a population data set), and providing proof-of-principle/proof-of-mechanism for a given therapeutic agent to block recruitment of dendritic cell precursors into brain of individuals patients who have or are at risk of developing neurodegenerative disease.

### BACKGROUND

Alzheimer's disease (AD) is estimated to afflict more than 20 million people worldwide and is believed to be the most common cause of dementia. AD is a disease characterized by degeneration and loss of neurons and also by the formation of senile plaques and neurofibrillary tangles. Recent human genetic studies indicate that neurodegeneration in AD is caused, at least in part, by chronic neuroinflammation. Presently, treatment of Alzheimer's disease is limited to the treatment of its symptoms rather than the underlying causes. Symptom-improving agents approved for this purpose include, for example, N-methyl-D-aspartate receptor antagonists such as memantine (Namenda.®., Forest Pharmaceuticals, Inc.), cholinesterase inhibitors such as donepezil (Aricept®, Pfizer), rivastigmine (Exelon®, Novartis), galantamine (Razadyne Reminyl®), and tacrine (Cognex®). New approaches for treating disease symptoms and more significantly for slowing or halting neurodegeneration to slow or halt disease progression are clearly needed.

Evidence gathered over several decades has documented a prominent, ubiquitous association of innate immune cells with pathological hallmarks of AD (Itagaki et al., 1989; Shen et al., 2013). Recent genome wide association studies (GWAS) show that variants of genes coding innate immune components are associated with elevated risk of AD, indicating that innate immune cells indeed have a causal role in the initiation and/or progression of disease (Bertram and Tanzi, 2009; Zhang et al., 2013). During normal brain development, innate immune mechanisms shape synaptic connections and carry out programmed neuronal death. In AD, however, inappropriate re-activation of this developmental program leads to synaptic dysfunction and neuronal loss that underlies disease symptoms and progression (Hong et al., 2016; Schafer et al., 2012). Therefore, blocking these aberrant immune system activities has therapeutic benefit in AD.

Dendritic cells comprise a type of innate immune cell best characterized for involvement in antigen presentation (Villani et al., 2017). In response to tissue damage or pathogen invasion, dendritic cells migrate from the blood stream into the damaged/pathogen-invaded tissue to mediate the initial innate immune system damage control response. Subsequently, through their involvement in antigen presentation, dendritic cells serve as a key intermediary between the innate and adaptive immune systems. However, in chronic inflammatory disease such as atherosclerosis, psoriasis, and lung fibrosis, innate immune mechanisms including those mediated by dendritic cells fail to re-establish homeostasis and their continued activation manifest as the deleterious effects of chronic inflammation. It has been assumed that dendritic cells do not migrate from the blood stream into the brain because of restriction imposed by the blood-brain-barrier. However, we provide compelling evidence that dendritic cells do indeed traffic from blood into brain in response to brain damage, notably the senile plaque and neurofibrillary tangle pathologies characteristic of AD, where they contribute to the deleterious effects of chronic neuroinflammation. Thus, measuring the trafficking of dendritic cells into the brain provides a method of detecting neuroinflammation, and blocking dendritic cell recruitment in chronic neuroinflammatory diseases such as AD is of therapeutic benefit.

### BRIEF DESCRIPTION OF THE DRAWINGS

These and other features, aspects, and advantages of the disclosure and disclosed embodiments will become better understood when the following detailed description is read with reference to the accompanying drawings in which like characters represent like parts throughout the drawings, wherein:

FIGS. 1A1, 1A2, 1B1, 1B2, 1C, 1D, 1E1 and 1E2 are a schematic illustration of a flow cytometry-based assay and illustrations of the data from the assay;

FIGS. 2A-2E are a schematic illustration of the assay and illustrations of the data from Hippocampal slices prepared from APP/PS1 mice;

FIGS. 3A-3D are illustrations of the data from Hippocampal slices prepared from APP/PS1 mice;

FIGS. 4A1-4A3, 4B, 4C and 4D1-4D4 are illustrations of the data from quantified acute recruitment of CD11c-labeled dendritic cells into brain of Tg4510 tauopathy mice;

FIGS. 5A and 5B are a schematic illustration of an assay and images showing the results of recruited dendritic cells that can also be detected by imaging of near-infrared fluorescence in brain;

FIGS. 6A and 6B are images showing the results of ICG-labeled cells that are specifically recruited into brain of transgenic vs. wild-type mice; and

FIGS. 7A-7D are images showing the results of pre-injection of ribosomal toxin saporin conjugated to a CD11c mAb followed by ICG injection in APP/PS1 transgenic mice.

### SUMMARY

A pathological hallmark of AD is the presence of innate immune cells associated with amyloid/tau deposits, evidence for which has accumulated over decades (Shen et al., 2013). Furthermore, recent GWAS show that innate immune gene variants, including genes coding for TREM2, CD33, HLA-DR, Mef2C, CR1/CD35, FcεRIβ, and Ship1, elevate AD risk (Zhang et al., 2013) and AD risk factor APOE4 also has a role in regulating innate immune function. Together, these data strongly indicate that innate immune dysfunction plays a causal role in AD and, more specifically, that chronic neuroinflammation may be an effector in AD progression, just as chronic inflammation is an effector in conditions such as atherosclerosis, psoriasis, and lung fibrosis. Thus, targeting chronic neuroinflammation is emerging as a new therapeutic strategy for AD.

Dendritic cells recruited from blood to brain are innate immune mediators of neuroinflammation in AD: While most others have focused attention on resident microglia as the innate immune mediators of neuroinflammation-induced brain damage, we have amassed compelling evidence that dendritic cells, recruited from the periphery into the brain, are key neuroinflammatory mediators. Ultrastructural and immunohistochemical studies from the early 1990s indicate the presence of immune cells on amyloid plaques having a phenotype consistent with blood-derived dendritic cells (Eikelenboom et al., 1991; Wisniewski and Wegiel, 1991). Infiltration of unspecified innate immune cells across the blood-brain barrier had also previously been demonstrated in amyloid-depositing transgenic mice (Lebson et al., 2010; Stalder et al., 2005). More recently, genome wide expression studies (GWES) in samples from human AD patients show a strong upregulation of genes consistent with dendritic cells (Wes et al., 2014; Zhang et al., 2013). These genes include those listed above that have been identified as AD risk factors. In translational studies of genome-wide gene expression in amyloid-depositing APP/PS1 and tauopathy Tg4510 mouse models of AD, we found that, the “dendritic cell maturation” pathway was the top upregulated pathway and the “leukocyte extravasation pathway” was among the top 10 upregulated pathways of over 400 canonical pathways examined in both models (Nelson, R. B. et al. Similar patterns of altered innate immunity and hematopoietic cell recruitment develop in Aβ-depositing (APP/PS1) and tauopathy (Tg4510) transgenic models of Alzheimer pathology Program No. 126.17 2017 Neuroscience Meeting Planner. Washington, DENDRITIC CELL: Society for Neuroscience, 2017. Online). Furthermore, in both the amyloid-depositing APP/PS1 and tauopathy Tg4510 mouse models of AD disease biology we demonstrate cells positive for the dendritic cell marker CD11c to be prominently associated with pathology (Nelson, R. B. et al. Program No. 126.17 2017 Neuroscience Meeting Planner Online). This is the same dendritic cell marker associated with pathology in AD brain (Eikelenboom et al., 1991). The identity of CD11c+ cells as dendritic cells was confirmed in the AD mouse models by demonstration that they expressed several other dendritic cell markers, including Dectin-1, CD103, and MHC-II. Thus, the anatomical and genetic data from AD patients, recapitulated in two distinct mouse models of Alzheimer-relevant pathology, demonstrate that dendritic cells are specifically associated with AD pathology in brain. Taken together, these data suggested to us that, contrary to prevailing dogma, dendritic cells may traffic into the brain in response to AD pathology.

The significance of the present disclosure is demonstrating that dendritic cells are recruited from blood into brain in response to AD pathology. Cells in blood were non-specifically labeled with the membrane dye, DiO, in the AD mouse models (Example 1). While free DiO is rapidly cleared from blood, the dye intercalates into and thereafter remains in blood cell membranes. One or 2 days after dye administration, brain cells were isolated and analyzed by flow cytometry. We detected a population of cells positive for both DiO and CD11c (Example 1). Since DiO does not cross the blood brain barrier, these CD11c⁺ cells must have been DiO-labeled while in the blood and subsequently recruited into brain. Importantly, the accumulation of DiO⁺/CD11c⁺ cells was only observed in mice with AD pathology, and there were essentially no DiO⁺/CD11c⁻ cells detected. These data demonstrate that the dendritic cells present in AD brain associated with amyloid and tau pathology are recruited from the blood into the brain. We also demonstrate active dendritic cell recruitment in a mouse model of tauopathy, the Tg4510 mouse (Example 4).

Very similar results and conclusions are drawn from experiments in which dendritic cells are labeled in the blood with the tracking dye indocyanine green (ICG; Examples 5, 6, & 7). Dendritic cells that have taken up ICG from the blood are observed to accumulate in APP/PS1 mice but not WT mice of the same age (Example 6), indicating that dendritic cell recruitment occurs specifically in response to brain pathology. Furthermore, the ICG-labeled cells that accumulate in brain are confirmed to be dendritic cells because depletion of dendritic cells from the blood using a saporin-conjugated CD11c antibody eliminates ICG-labeled cell accumulation in APP/PS1 mice (Example 7).

Recruited innate immune cells are deleterious in AD: The dendritic cells recruited from the periphery into the brain in response to AD pathology account for the contribution of innate immune mediators in the progression of AD that were uncovered by GWAS (Malik et al., 2015) and including the work from the Stevens lab (Hong et al., 2016).

Innate immune cells in the blood are the “first responders” to tissue injury. Damage to organs result in signaling to the adjacent vasculature. Cell adhesion molecule expression on the vascular endothelium then attracts circulating cells, which become immobilized prior to “extravasating” across the endothelium into the tissue. Responding innate immune cells play two major roles: Surveillance for and destruction of invading pathogens, and initiation of tissue repair responses. However, if the innate immune response persists, chronic inflammation ensues with deleterious consequences, as is the case in atherosclerosis, psoriasis, lung fibrosis, and other chronic inflammatory conditions. In the brain, the tissue repair response is believed to be mediated exclusively by the brain's resident innate immune cells, the microglia, because blood-derived innate immune cells are presumed to be excluded by the blood brain barrier. However, as indicated above, we have demonstrated that dendritic cells are specifically recruited from the blood into brain in response to AD-relevant pathology. The mechanism by which these recruited dendritic cells mediate neuroinflammation-induced brain damage comes from recent discoveries illuminating a physiological function for innate immune mechanisms in brain development. Innate immune cells play an integral role in synapse pruning and remodeling during brain development (Schafer et al., 2012). Innate immune cells are also integral in the developmental process of programmed neuronal death (Wakselman et al., 2008). Together, these innate immune cell functions establish a basal neural circuitry comprising ˜10% of newborn neurons, and the orderly removal of the superfluous neurons (˜90% of those born) during early brain development. However, these developmental immune cell functions are inappropriately active in adult AD brain, which is highly deleterious. Studies using 2-photon microscopy in APP/PS1 mice reveal that synaptic spine turnover is greatly accelerated in the penumbral region of amyloid plaques (Bittner et al., 2012; Spires-Jones et al., 2007). We found dendritic cells accumulate in this penumbral region in spatial and temporal correlation with the appearance of dystrophic neurites in a similar transgenic mouse line (Nelson, R. B. et al. Program No. 126.17 2017 Neuroscience Meeting Planner Online). Moreover, we observed that synaptic transmission measured in hippocampal slices prepared from these mice was progressively impaired in temporal correlation with the accumulation of dendritic cells (Examples 2 & 3), as were disruption of normal patterns of brain activity measured using EEG (Nelson, R. B. et al. Program No. 126.17 2017 Neuroscience Meeting Planner Online). Thus, we show that recruited dendritic cells are the ‘smoking gun’ immune mediators that cause the deleterious synaptic turnover that ultimately leads to neuronal death in AD. This conclusion is consistent with recent work from Silver and colleagues indicating that immune cells recruited from the periphery, not resident microglia, are responsible for deleterious axonal dieback from the site of spinal cord injury (Evans et al., 2014). More significantly, precedent for the concept that immune cell recruitment can compromise CNS comes from relapsing-remitting multiple sclerosis, where the top 3 therapies for treating this disease share in common—via different mechanisms—the inhibition of immune cell trafficking into brain.

Methods for evaluating dendritic cell recruitment into brain of transgenic mouse models of AD-like pathology: The in vivo assays schematized in Examples 1 and 5 track dendritic cells infiltrating the brain in response to AD-relevant pathologies in the APP/PS1 and Tg4510 mice as well as in humans suffering AD-related pathologies. When used in animal models of AD such as the APP/PS1 and Tg4510 mouse lines, these assays provide a rapid means to test drug development candidates for their potential ability to block dendritic cell recruitment. There are several novel aspects of these assays that merit specific consideration. Our model uses the non-specific fluorescent dyes DiO or indocyanine green to label all circulating cells, and then flow cytometry of isolated brain cells or infrared imaging of hemi-brains, respectively, to identify recruited cells. These methods approach the measurement of recruitment using a pulse/chase design. The unbiased nature of these methods also proves advantageous in revealing the unique biology of the recruited cells. The method is amenable to using a broad panel of antibodies against various innate immune cell types selected based on GWES expression patterns to characterize DiO+ cells in brain (i.e., that originated from blood). These analyses revealed that the sole population entering the brain in the window of our pulse-chase design in the APP/PS1 and Tg4510 mice expressed a constellation of markers consistent with dendritic cells. Significantly, the set of markers (CD11c and others) expressed by the cells using these methods has been largely ignored in the AD literature after the early work of Eikelenboom and Wisniewski noted above. In contrast, conventional protocols typically deplete endogenous bone marrow-derived macrophages (BMDMs) by irradiation, then introduce adoptively transferred BMDMs tagged with a specific expression vector. The harsh treatments used to deplete BMDMs often induce artefactual recruitment through vascular inflammation (Kierdorf et al., 2013). The methods described in the present disclosure, which label non-specifically all the endogenous hematopoietic cells and tracks acute recruitment, avoids harsh treatments known to confound studies of recruitment by inducing recruitment of immune cells to the harsh experimental treatment rather than or in addition to the disease biology under study. These ‘false positive’ recruited immune cells could lead to erroneous identification of biomarkers and therapeutic agents.

Dendritic cell recruitment from blood to brain as a biomarker for neuro-inflammation in AD: No clinically validated biomarkers currently exist for the progression of neuroinflammatory pathology in AD or other neurodegenerative, neurological, or neuropsychiatric disease or disorder. Such a biomarker would be useful for disease diagnosis and for staging the progression of disease. In clinical trials, such a biomarker would be useful for patient selection and stratification, especially if it is found that certain sub-types of AD or other neurodegenerative disease have as a prominent component of pathology the recruitment of dendritic cell precursors from blood into brain. Such a biomarker would also be useful as an outcome measure for therapeutics that aim to reduce neuroinflammation in general, and as an outcome measure for any therapeutic that modifies underlying disease pathology that in turn is the trigger for the neuroinflammatory response. In AD, a biomarker of immune cell recruitment would be particularly useful in the context of other biomarkers for AD pathology, such as PiB and other amyloid imaging agents, tau imaging agents, TSPO and related microglial activation markers.

The present disclosure encompasses translation of the dendritic cell tracking methodologies that we developed in AD mouse models into human patients as an index of ongoing chronic neuroinflammation (Example 5). This biomarker of neuroinflammation has use in clinical trials for neurodegenerative diseases, such as Alzheimer's disease, small vessel diseases that include cerebral amyloid angiopathy, and fronto-temporal dementias. As one example, dendritic cells may be labeled in the blood with indocyanine green (IC-GREEN®) and later detected in association with amyloid plaques by near infrared spectroscopy (NIRS). IC-GREEN® is a fluorescent dye for which the excitation/emission spectrum resides in the near infrared range. IC-GREEN® has been used extensively in humans for over 60 years as an agent to assess cardiac output and hepatic blood flow, and for ophthalmic angiography. This agent is considered very safe, with incidence of adverse events fewer than 1/40,000. We have demonstrated in an amyloid mouse model that IC-GREEN® administered systemically accumulates in dendritic cells and, in mice with amyloid pathology, these labeled cells accumulate in the brain over 24-48 in association with amyloid pathology and that this accumulation is detectable with near infrared spectroscopy (Examples 5-7). Intravenous infusion of IC-GREEN® to label dendritic cells in humans while these cells circulate in the blood may be detected in human brain of AD patients using non-invasive near infrared spectroscopy, analogous to our technique developed in the AD mouse models. Quantifying recruitment of labeled cells into the human cortex of patients suffering AD will be accomplished using portable near-infrared spectroscopy (NIRS) technology (Abtahi, M., Cay, G., Saikia, M. J., & Mankodiya, K. (2016), Designing and testing a wearable, wireless fNIRS patch. 38th Annual International Conference of the IEEE Engineering in Medicine and Biology Society (EMBC). doi:10.1109/embc.2016.7592168). NIRS is a noninvasive optical measurement technique that takes advantage of the fact that NIR light (700-900 nm) is not absorbed by skin, tissue, bone and lipids. However, because hemoglobin is a strong absorber, the technique can be used to monitor cerebral blood flow and is currently used for monitoring the hemodynamic activity of brain in healthy volunteers performing cognitive and motor tasks as well as in patients with Parkinson's disease. Given that we detect cells labeled with a highly fluorescent infrared dye, the technology has more than sufficient sensitivity to detect dendritic cells recruited from the blood to cortex in humans.

Another aspect of the present disclosure includes modifying methods that currently exist for detecting and tracking hematopoietic immune cells into multiple peripheral tissues. These methods can be modified to detect the selective migration of dendritic cell precursors from blood into brain as a marker of human neurodegenerative disease. The present disclosure describes such methods that can be used to evaluate and compare different embodiments of these methods in vivo for their relative sensitivity and feasibility in detecting the recruitment of dendritic cell precursors into the brains of transgenic mice overexpressing various mutations associated with induction of distinct neurodegenerative disease pathologies. The present disclosure further describes methods for temporally correlating brain pathologies and deficits in brain function with the recruitment of dendritic cells into the brains of these transgenic mice.

Another aspect of the present disclosure relates to labeling of isolated peripheral blood mononuclear cells from patients using paramagnetic particles, followed by their intravenous re-introduction into the patients, and tracking over time of signal uptake into tissues using in vivo structural MR imaging. MR imaging of brain is well-established and should therefore allow the tracking of paramagnetic bead-bearing cells into the brain (cf. Hoogeven et al., 2017).

Another aspect of the present disclosure also relates to labeling of isolated peripheral blood mononuclear cells from patients using the SPECT ligand 99mTc-HMPAO, followed by their intravenous re-introduction into patients, and tracking over time of signal uptake into atherosclerotic lesions in heart tissues using in vivo SPECT imaging (van der Valk et al., 2014; Hoogeven et al., 2017). SPECT-based imaging of brain is well-established and should therefore allow the tracking of SPECT ligand-bearing cells into the brain.

Additional aspects include attaching imaging moieties to monoclonal antibodies that are specific for dendritic cell precursors in blood and allowing such modified antibodies to selectively adhere to dendritic cell precursors circulating in blood, and tracking the uptake of these labeled cells into brain by matching the specific imaging reporter tag on the antibody to the appropriate imaging modality to be used.

Methods of blocking dendritic cell recruitment into brain for treatment and prevention of neurodegenerative disease: Based on our demonstration of dendritic cell recruitment from blood to brain in different models of neurodegenerative disease, the elevated risk of AD associated with variants of multiple genes that have preferential expression in dendritic cells, the temporal correlation of dendritic cell recruitment in brain with development of anatomical pathology and functional deficits, and the spatial association of dendritic cells in brain with pathological hallmarks of AD and disruption of synaptic spine circuitry associated with these hallmarks, dendritic cells are identified as a key mediator of the neuroinflammation that links AD pathology to the synaptic dysfunction and neuronal death that underlies AD symptoms and disease progression. Thus, blocking dendritic cell recruitment into AD brain will reduce neuroinflammation to ameliorate AD symptoms and slow or halt disease progression. The present disclosure includes target mechanisms (including agents, such as compounds, known to affect those mechanisms) previously associated with dendritic cell recruitment in peripheral diseases for their previously unknown and unanticipated potential as a therapeutic to reduce neuroinflammation in AD and thereby reduce symptoms of AD and slow or halt disease progression. The present disclosure also provides the methods for measuring dendritic cell recruitment into brain that are detailed herein as a selection of enablements for such mechanisms in their specific utility for neurodegenerative diseases modeled by the transgenic mouse models described. For therapeutic purposes, “agents” or “therapeutic agents” (such as compounds) as used herein refers to pharmaceutical materials that reduce or block dendritic cell recruitment across the blood brain barrier. Although blockage (e.g. reduce to zero) is desirable, pharmacologically reducing recruitment by 50% may lead to an efficacious result.

The mechanisms listed below for targeting by potential therapeutic agents are selected for a previously unknown and unsuspected therapeutic use in the treatment of AD based on their established role in dendritic cell migration into tissues, dendritic cell maturation, or dendritic cell signaling, all of which contribute to the pathological process whereby dendritic cells are recruited from blood into brain in AD to mediate chronic neuroinflammation. Of particular interest are mechanisms related to dendritic cell function that confer risk for developing AD as identified in human genetic studies:

A first class includes dendritic cell receptors that are implicated in dendritic cell recruitment and that have an increased expression on innate immune cells associated with AD pathology. Examples include but are not limited to CR4 (CD11c/CD18), Dectin 1 (Clec7a), CSF1R (M-CSFR), Galectin 3, and TREM2. Agents known to affect these mechanisms are known and these agents, including compounds, may now be used in conjunction with the methods described herein to treat and monitor the progression of AD.

A second class includes enzymes that regulate fibrinogen/fibrin processing and/or tissue deposition, and/or unmasking of CR3 and/or CR4 dendritic cell-binding domains. Examples include but are not limited to Factor XIa/XIIa, Factor Xa, thrombin, and P2Y12R. Agents known to affect these enzymes are known and these agents, such as compounds, may now be used in conjunction with the methods described herein to treat and monitor the progression of AD.

A third class includes dendritic cell receptors implicated in dendritic cell motility, recruitment, and/or activation, but not reported to date to be present on cells found in AD brain. Examples include but are not limited to CCR7, DC-SIGN, CRTH2, and P2X7R. Agents known to affect these receptors are known and these agents, such as compounds, may now be used in conjunction with the methods described herein to treat and monitor the progression of AD.

A fourth class includes ion channels that regulate inflammatory phenotype that are expressed by and have a function associated with dendritic cells. One example is KCNN4, but other examples have also been described. Agents known to affect these ion channels are known and these agents, such as compounds, may now be used in conjunction with the methods described herein to treat and monitor the progression of AD.

A fifth class includes enzymes that regulate inflammatory phenotype in innate immune cells, and are expressed by and have a funciton associated with dendritic cells. Examples include but are not limited to Arg1, Arg2, KMO, PDE4, PDE8, and MEK. Agents known to affect these enzymes are known and these agents, such as compounds, may now be used in conjunction with the methods described herein to treat and monitor the progression of AD.

A sixth class includes vascular adhesion molecules known to be upregulated on vascular endothelium and to mediate dendritic cell endothelial trans-migration. Examples include but are not limited to Sema4D/7A, ICAM-2, ALCAM, PECAM, and VCAM. Agents known to affect vascular adhesion are known and these agents, such as compounds, may now be used in conjunction with the methods described herein to treat and monitor the progression of AD.

A seventh class includes micro-RNAs known to regulate dendritic cell phenotypic fate and dendritic cell receptor expression pattern. Examples include but are not limited to miR-155 and miR-511. Agents known to affect these RNA's are known and these agents, such as compounds, may now be used in conjunction with the methods described herein to treat and monitor the progression of AD.

An eighth class includes receptor tyrosine kinases known to regulate dendritic cell phenotype and dendritic cell receptor expression pattern. Examples include but are not limited to Flt3, MerTK, EphA2, EphB2, Tyro3, AxI, and Mer. Agents known to affect receptor tyrosine kinases are known and these agents, such as compounds, may now be used in conjunction with the methods described herein to treat and monitor the progression of AD.

In one embodiment, the aspects of the present disclosure are directed to a method, e.g., a clinical method, of measuring or quantifying dendritic cell migration into brain and to select and stratify suitably responsive individuals (patients) for inclusion or exclusion in a clinical trial for the treatment of a neurodegenerative disease.

In another embodiment, the aspects of the present disclosure are directed to a method of measuring or quantifying dendritic cell migration into brain, e.g., in a clinical setting, in concert with one or more other biomarkers, to diagnose, chronic and/or acute stages of neurodegenerative disease progression in individual patients (or as an aggregate population data set).

In another embodiment, the aspects of the present disclosure are directed to a method of measuring or quantifying dendritic cell migration into a brain, across the blood brain barrier, to provide a proof-of-principle/proof-of-mechanism measure for, e.g., clinical, biological, disease modifying assessment of the ability (inhibitory, modulating, modifying, preventing, treating) of a therapeutic agent to block recruitment of dendritic cell precursors into brain.

In another embodiment, the aspects of the present disclosure are directed to a method of labeling peripheral blood mononuclear cells using an IV infusion of indocyanine green (IC-GREEN®) to label said cells in the body and measuring over time signal uptake into brain using near infrared spectroscopy/imaging of the brain.

In another embodiment, the aspects of the present disclosure are directed to a method of labeling isolated peripheral blood mononuclear cells with paramagnetic particles, re-infusing said cells, and measuring over time signal uptake into brain using in vivo structural MR imaging.

In another embodiment, the aspects of the present disclosure are directed to a method of labeling isolated peripheral blood mononuclear cells with 99mTc-HMPAO prior to re-infusion, and measuring over time signal uptake into brain using in vivo hybrid SPECT/CT imaging.

In another embodiment, the aspects of the present disclosure are directed to a method of introducing monoclonal antibodies that are specific for dendritic cell precursors into blood, allowing adherence to target circulating immune cells, and measuring uptake into brain matching appropriate imaging reporter tags on the antibodies to the imaging modality to be used.

In another embodiment, the aspects of the present disclosure are directed to a method of using single cell transcriptomics of whole blood to detect a specific dendritic cell population(s) decreased in blood of individuals with neurodegenerative disease (e.g. due to recruitment in brain), using qPCR as a diagnostic to indirectly measure dendritic cell precursor depletion.

In another embodiment, the aspects of the present disclosure are directed to a method of using a panel of monoclonal antibodies of whole blood to detect a specific dendritic cell population(s) decreased in blood of individuals with neurodegenerative disease (e.g. due to recruitment in brain) and using ELISA as a diagnostic to indirectly measure dendritic cell precursor depletion.

In another embodiment, the aspects of the present disclosure are directed to a method of identifying efficacious compounds (e.g. including optimizing dosage) that suppress or block recruitment of dendritic cell precursors into the brains of transgenic mice overexpressing various mutations associated with induction of distinct neurodegenerative disease pathologies and phenotypes.

In another embodiment, the aspects of the present disclosure are directed to a method of identifying a particular subset of dendritic cell precursors migrating into brain of animal disease models and human patients.

In another embodiment, the aspects of the present disclosure are directed to a method of evaluating agents for their relative efficacy to block recruitment of dendritic cell precursor migration into brain in the context of neurodegenerative disease. In another embodiment, the aspects of the present disclosure are directed to a method where the agents include the mechanistic classes of agents of the embodiments exemplified herein.

In another embodiment, the aspects of the present disclosure are directed to a method of treating neurodegenerative disorders in a mammal (e.g. including a human) in need of such treatment comprising administering to said mammal a therapeutically effective amount of a dendritic cell migration inhibiting or blocking agent. In another embodiment, the aspects of the present disclosure are directed to a method where the neurodegenerative disorder is selected from Alzheimer's disease; Parkinson's disease; brain injury; stroke; and cerebrovascular disease.

The present application claims the benefit of priority to U.S. Provisional Patent Application No. 62/583,959, filed Nov. 9, 2017, which is incorporated herein by reference in its entirety.

### DETAILED DESCRIPTION

The methods of the present disclosure involve measuring recruitment and migration of peripheral dendritic cells into the brain, across the blood brain barrier. These assays provide a framework to assess the status of AD in an individual as well as in the aggregate a baseline of AD populations. These assays also provide a basis to identify an efficacious dose for an individualized AD therapy as well as monitoring the effect of treatment through time.

References describing these aforesaid mechanisms and compounds include the following:

Bero, A. W., A. Q. Bauer, F. R. Stewart, B. R. White, J. R. Cirrito, M. E. Raichle, J. P. Culver, and D. M. Holtzman. 2012. Bidirectional relationship between functional connectivity and amyloid-beta deposition in mouse brain. The Journal of neuroscience : the official journal of the Society for Neuroscience. 32:4334-4340.

Bertram, L., and R. E. Tanzi. 2009. Genome-wide association studies in Alzheimer's disease. Human Molecular Genetics. 18:R137-R145.

Bittner, T., S. Burgold, M. M. Dorostkar, M. Fuhrmann, B. M. Wegenast-Braun, B. Schmidt, H. Kretzschmar, and J. Herms. 2012. Amyloid plaque formation precedes dendritic spine loss. Acta neuropathologica. 124:797-807.

Buckner, R. L., J. Sepulcre, T. Talukdar, F. M. Krienen, H. Liu, T. Hedden, J. R. Andrews-Hanna, R. A. Sperling, and K. A. Johnson. 2009. Cortical hubs revealed by intrinsic functional connectivity: mapping, assessment of stability, and relation to Alzheimer's disease. The Journal of neuroscience : the official journal of the Society for Neuroscience. 29:1860-1873.

Colton, C. A. 2013. Immune heterogeneity in neuroinflammation: dendritic cells in the brain. Journal of neuroimmune pharmacology : the official journal of the Society on Neurolmmune Pharmacology. 8:145-162.

Eikelenboom, P., J. M. Rozemuller, G. Kraal, F. C. Stam, P. A. McBride, M. E. Bruce, and H. Fraser. 1991. Cerebral amyloid plaques in Alzheimer's disease but not in scrapie-affected mice are closely associated with a local inflammatory process. Virchows Archiv. B, Cell pathology including molecular pathology. 60:329-336.

El Khoury, J., M. Toft, S. E. Hickman, T. K. Means, K. Terada, C. Geula, and A. D. Luster. 2007. Ccr2 deficiency impairs microglial accumulation and accelerates progression of Alzheimer-like disease. Nature medicine. 13:432-438.

Evans, T. A., D. S. Barkauskas, J. T. Myers, E. G. Hare, J. Q. You, R. M. Ransohoff, A. Y. Huang, and J. Silver. 2014. High-resolution intravital imaging reveals that blood-derived macrophages but not resident microglia facilitate secondary axonal dieback in traumatic spinal cord injury. Experimental neurology. 254:109-120.

Finnema, S. J., N. B. Nabulsi, T. Eid, K. Detyniecki, S. F. Lin, M. K. Chen, R. Dhaher, D. Matuskey, E. Baum, D. Holden, D. D. Spencer, J. Mercier, J. Hannestad, Y. Huang, and R. E. Carson. 2016. Imaging synaptic density in the living human brain. Science translational medicine. 8:348ra396.

Hong, S., V. F. Beja-Glasser, B. M. Nfonoyim, A. Frouin, S. Li, S. Ramakrishnan, K. M. Merry, Q. Shi, A. Rosenthal, B. A. Barres, C. A. Lemere, D. J. Selkoe, and B. Stevens. 2016. Complement and microglia mediate early synapse loss in Alzheimer mouse models. Science. 352:712-716.

Itagaki, S., P. L. McGeer, H. Akiyama, S. Zhu, and D. Selkoe. 1989. Relationship of microglia and astrocytes to amyloid deposits of Alzheimer disease. Journal of neuroimmunology. 24:173-182.

Kan, M. J., J. E. Lee, J. G. Wilson, A. L. Everhart, C. M. Brown, A. N. Hoofnagle, M. Jansen, M. P. Vitek, M. D. Gunn, and C. A. Colton. 2015. Arginine deprivation and immune suppression in a mouse model of Alzheimer's disease. The Journal of neuroscience : the official journal of the Society for Neuroscience. 35:5969-5982.

Kierdorf, K., N. Katzmarski, C. A. Haas, and M. Prinz. 2013. Bone Marrow Cell Recruitment to the Brain in the Absence of Irradiation or Parabiosis Bias. PloS one. 8:e58544.

Lebson, L., K. Nash, S. Kamath, D. Herber, N. Carty, D. C. Lee, Q. Li, K. Szekeres, U. Jinwal, J. Koren, C. A. Dickey, P. E. Gottschall, D. Morgan, and M. N. Gordon. 2010. Trafficking CD11b-positive blood cells deliver therapeutic genes to the brain of amyloid-depositing transgenic mice. The Journal of neuroscience: the official journal of the Society for Neuroscience. 30:9651-9658.

Malik, M., I. Parikh, J. B. Vasquez, C. Smith, L. Tai, G. Bu, M. J. LaDu, D. W. Fardo, G. W. Rebeck, and S. Estus. 2015. Genetics ignite focus on microglial inflammation in Alzheimer's disease. Molecular Neurodegeneration. 10:1-12.

Phinney, A. L., T. Deller, M. Stalder, M. E. Calhoun, M. Frotscher, B. Sommer, M. Staufenbiel, and M. Jucker. 1999. Cerebral amyloid induces aberrant axonal sprouting and ectopic terminal formation in amyloid precursor protein transgenic mice. The Journal of neuroscience: the official journal of the Society for Neuroscience. 19:8552-8559.

Piras, A., L. Collin, F. Grüninger, C. Graff, and A. Rönnbäck. 2016. Autophagic and lysosomal defects in human tauopathies: analysis of post-mortem brain from patients with familial Alzheimer disease, corticobasal degeneration and progressive supranuclear palsy. Acta Neuropathologica Communications. 4:22.

Schafer, D. P., E. K. Lehrman, A. G. Kautzman, R. Koyama, A. R. Mardinly, R. Yamasaki, R. M. Ransohoff, M. E. Greenberg, B. A. Barres, and B. Stevens. 2012. Microglia sculpt postnatal neural circuits in an activity and complement-dependent manner. Neuron. 74:691-705.

Shen, Y., L. Yang, and R. Li. 2013. What does complement do in Alzheimer's disease? Old molecules with new insights. Translational Neurodegeneration. 2:21-21.

Spires-Jones, T. L., M. Meyer-Luehmann, J. D. Osetek, P. B. Jones, E. A. Stern, B. J. Bacskai, and B. T. Hyman. 2007. Impaired spine stability underlies plaque-related spine loss in an Alzheimer's disease mouse model. The American journal of pathology. 171:1304-1311.

Stalder, A. K., F. Ermini, L. Bondolfi, W. Krenger, G. J. Burbach, T. Deller, J. Coomaraswamy, M. Staufenbiel, R. Landmann, and M. Jucker. 2005. Invasion of hematopoietic cells into the brain of amyloid precursor protein transgenic mice. The Journal of neuroscience : the official journal of the Society for Neuroscience. 25:11125-11132.

van der Valk, F. M., J. Kroon, W. V. Potters, R. M. Thurlings, R. J. Bennink, H. J. Verberne, A. J. Nederveen, M. Nieuwdorp, W. J. Mulder, Z. A. Fayad, J. D. van Buul, and E. S. Stroes. 2014. In vivo imaging of enhanced leukocyte accumulation in atherosclerotic lesions in humans. Journal of the American College of Cardiology. 64:1019-1029.

Wakselman, S., C. Bechade, A. Roumier, D. Bernard, A. Triller, and A. Bessis. 2008. Developmental neuronal death in hippocampus requires the microglial CD11b integrin and DAP12 immunoreceptor. The Journal of neuroscience: the official journal of the Society for Neuroscience. 28:8138-8143.

Wes, P. D., A. Easton, J. Corradi, D. M. Barten, N. Devidze, L. B. DeCarr, A. Truong, A. He, N. X. Barrezueta, C. Polson, C. Bourin, M. E. Flynn, S. Keenan, R. Lidge, J. Meredith, J. Natale, S. Sankaranarayanan, G. W. Cadelina, C. F. Albright, and A. M. Cacace. 2014. Tau Overexpression Impacts a Neuroinflammation Gene Expression Network Perturbed in Alzheimer's Disease. PloS one. 9:e106050.

Wisniewski, H. M., and J. Wegiel. 1991. Spatial relationships between astrocytes and classical plaque components. Neurobiol Aging. 12:593-600.

Zhang, B., C. Gaiteri, L.G. Bodea, Z. Wang, J. McElwee, A. A. Podtelezhnikov, C. Zhang, T. Xie, L. Tran, R. Dobrin, E. Fluder, B. Clurman, S. Melquist, M. Narayanan, C. Suver, H. Shah, M. Mahajan, T. Gillis, J. Mysore, M. E. MacDonald, J. R. Lamb, D. A. Bennett, C. Molony, D. J. Stone, V. Gudnason, A. J. Myers, E. E. Schadt, H. Neumann, J. Zhu, and V. Emilsson. 2013. Integrated systems approach identifies genetic nodes and networks in late-onset Alzheimer's disease. Cell. 153:707-720.

### EXAMPLES

The following examples demonstrate methods that enabled measuring the recruitment of dendritic cells into brain for the purposes of screening for therapeutic agents that block dendritic cell recruitment in animal models and in humans, and for the diagnosis of dendritic cell recruitment as a biomarker of neuroinflammation in humans suffering neurodegenerative conditions, notably AD.

### Example 1

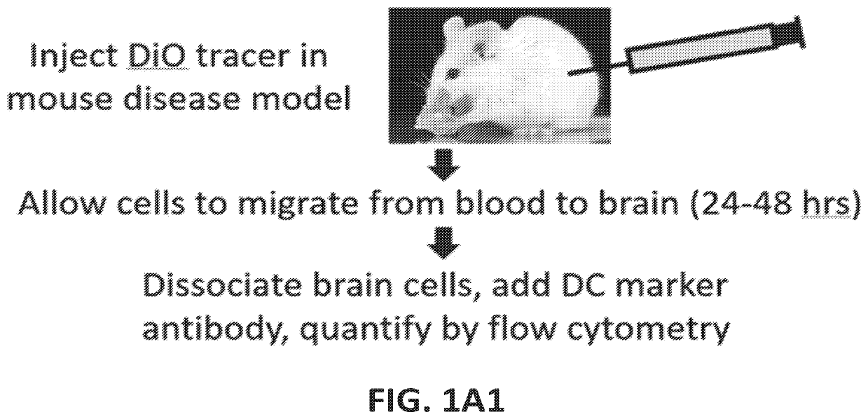

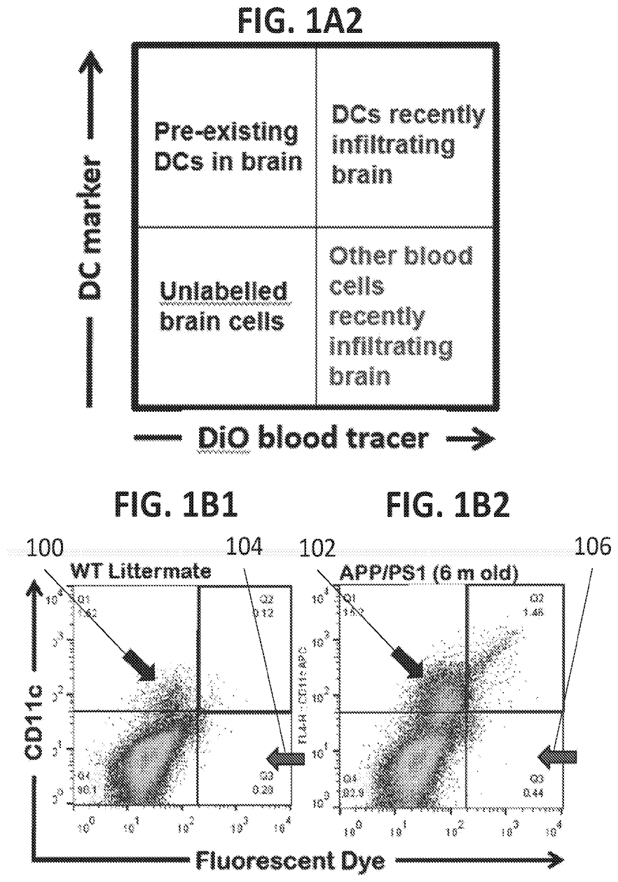

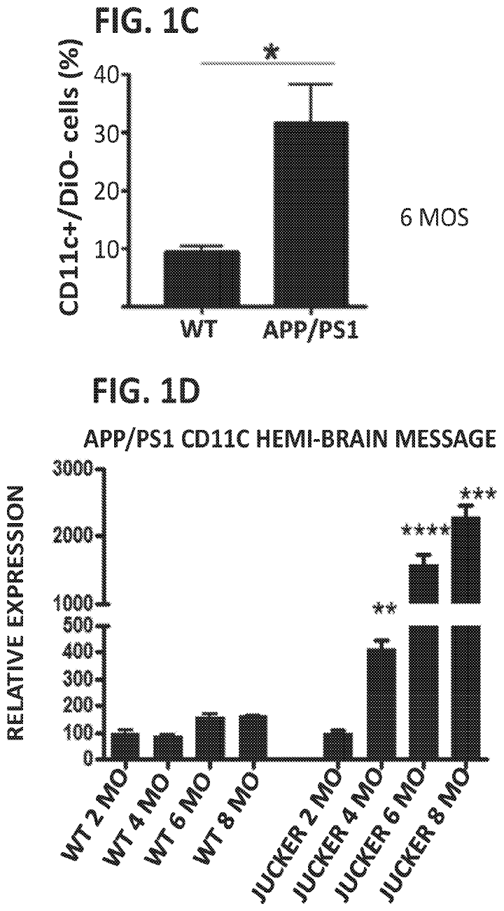

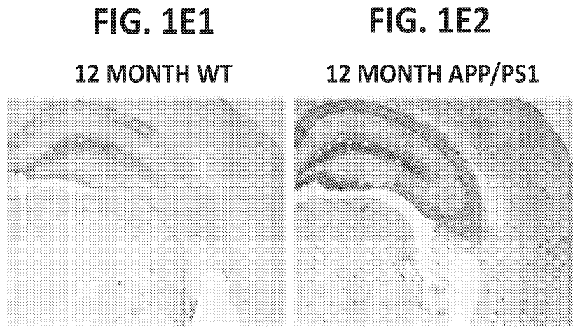

### We Established a Flow Cytometry-Based Assay (FIG. 1A1) Able to Demonstrate Acute Recruitment of Dendritic Cells into Brain, Quantify the Resident Population of Dendritic Cells at Any Given Age, and Identify CD11c-Labeled Dendritic Cells as the Major Population of Recruited Cells in APP/PS1 Mice

A) FIG. 1A2: Schematic of cell populations identified by our flow cytometry-based assay. Cells isolated from brains of mice administered 48 hrs. earlier with the rapidly cleared, non-brain penetrant membrane intercalating dye DiO are identified for the presence of a dendritic cell marker (y-axis) or DiO (x-axis). Cell number is represented in the form of a color code. Cells in the upper left quadrant are dendritic cells that had accumulated into brain prior to DiO administration. Cells in the upper right quadrant are dendritic cells labeled with DiO in the blood stream and that have migrated into brain in the 48 hrs. after DiO administration. Cells in the lower left quadrant are resident brain cells that are not dendritic cells. Cells in the lower right quadrant are any other cells that are not dendritic cells but that were labeled with DiO in the blood stream and that have migrated into brain in the 48 hrs. after DiO administration

B) FIGS. 1B1 and 1B2: “WT Littermate” includes the result from 6 month old wild-type mice that were litter mates to “APP/PS1 (6 mos old)”, which includes the result from 6 month old APP/PS1 transgenic mice. By non-specifically labeling blood-borne cells with the fluorescent membrane tracer DiO and using a “pulse-chase” design, we discriminate pre-existing dendritic cell populations in the brain (i.e. present prior to “pulse-labeling”, upper left quadrant). Note that this dendritic cell population is much larger in the APP/PS1 AD mouse model than in WT litter mates. Dendritic cell marker+/DiO+ double-labeled cells (upper right quadrant) are dendritic cells that have infiltrated the brain during the “chase” period. Note that these cells are present in the APP/PS1 mouse samples but are absent from the WT sample. Flow cytometry showed a selective accumulation of CD11c-labeled cells in the brains of APP/PS1 transgenic mice relative to wild-type mice. These were cells present in brain prior to DiO injection (blue arrows labelled 100 in FIGS. 1B1 and 102 in FIG. 1B2). Flow cytometry also confirmed_that in neither APP/PS1 nor WT samples was there a population of DiO-positive cells in brain that were NOT labeled with the dendritic cell marker, indicating that there were no other major non-dendritic cell populations of recruited cells during the DiO labeling period (green arrows labelled 104 in FIGS. 1B1 and 106 in FIG. 1B2).

C) FIG. 1C: The increase in DiO⁺/CD11c⁺ cells in samples from the APP/PS1 mice relative to the WT mice is indicated.

D) FIG. 1D: The relative expression levels of CD11c mRNA (gene designation is “Itgax”) increase in the brain of APP/PS1 mice over months of age—2, 4, 6, and 8 mos old mice were compared-in correlation with the increase in amyloid pathology. No such increase in CD11c mRNA occurs in age-matched WT mice.

E) FIGS. 1E1 and 1E2: “12 m WT” includes the result from 12 month old wild-type mice that were litter mates to 12 month old APP/PS1 transgenic mice, and “12 m APPPS1” includes the result from 12 month old APP/PS1 transgenic mice. Cells labeled for the DC-enriched marker CD11c, measured by immunohistochemistry, selectively increase as a function of age and transgene in the brains of APP/PS1 A□-depositing mice. The increase in immunolabeling is paralleled by increases in CD11c message (D above). Significantly, CD11c immunolabeling is concentrated near amyloid pathology, as shown in a sample from 12 mos old mouse. CD11c immunolabeling was low to undetectable in age-matched WT mice. Representative immunohistochemical images are shown.

### Example 2

### Hippocampal Slices Prepared from APP/PS1 Mice Show Progressive Deficits in Synaptic Transmission that Correlate Over Months of Age with the Increase in Amyloid Plaque Load

While cognitive impairments in APP/PS1 mice are often subtle to measure, physiological deficits measured ex vivo are a sensitive means for detecting changes in neural circuits associated with amyloid plaque deposition.

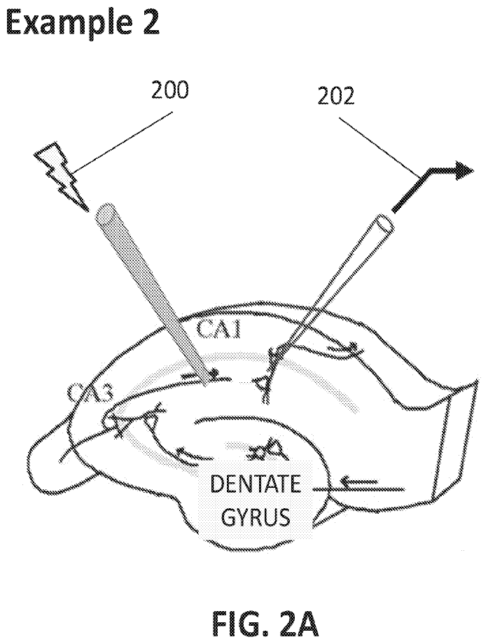

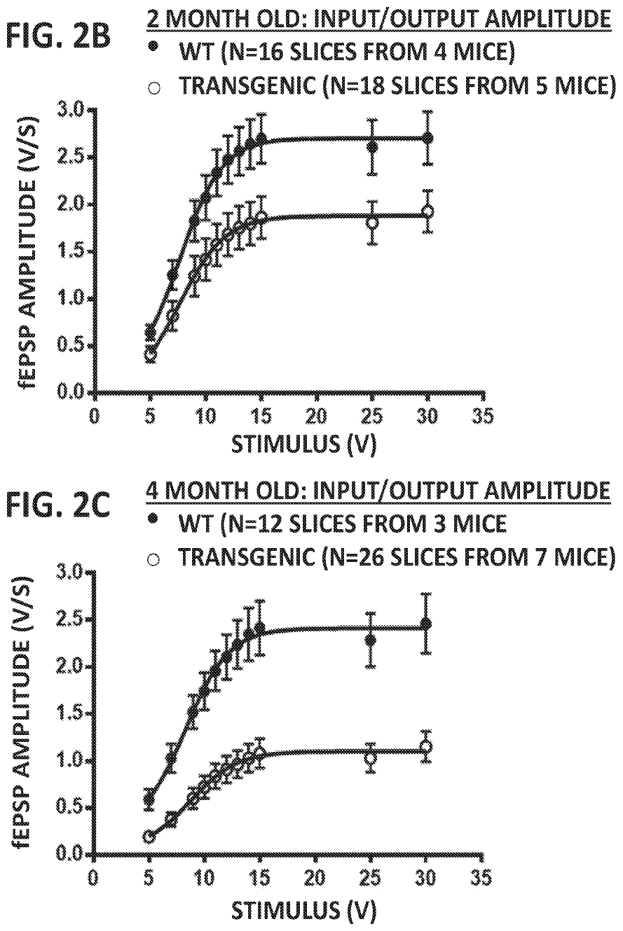

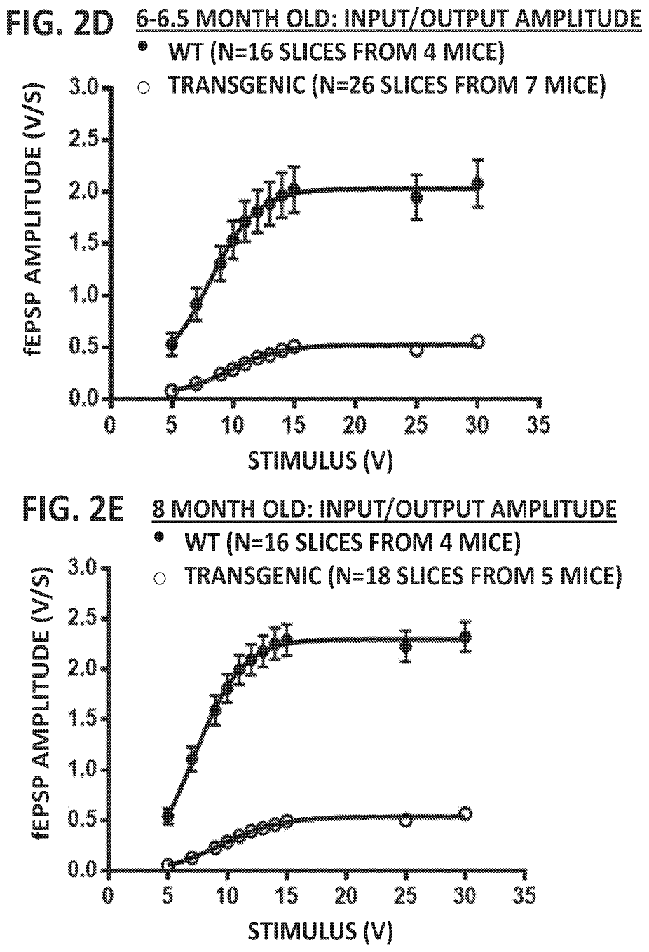

A) FIG. 2A: Schematic of hippocampal slice preparation. The preparation and electrical stimulation of hippocampal slices 200 and recording of synaptic responses by electrophysiology 202 is a well established technique widely familiar to those skilled in the art. Field potentials, a measure of synaptic transmission, were recorded from the CA1 region of hippocampus in response to stimulation of the Shaffer collateral inputs.

B) FIG. 2B: CA1 region field potentials were lower in slices from 2-mos-old APP/PS1 mice compared to slices taken from age-matched WT mice.

C) FIG. 2C: CA3 region field potentials were even lower in slices from 4-mos-old APP/PS1 mice compared to slices taken from age-matched WT mice.

D) and E) FIGS. 2D and 2E: CA3 region field potentials were almost absent in slices from APP/PS1 mice older than 6 months compared to slices taken from age-matched WT mice.

### Example 3

### Hippocampal Slices Prepared from APP/PS1 Mice also Evidence Progressive Deficits in Hippocampal Long-Term Potentiation (LTP) that are Correlated to the Increase in Plaque Load with Age

LTP is widely believed to represent a form of synaptic plasticity related to learning and memory. Measurement of LTP by electrophysiology is a well-established technique widely familiar to those skilled in the art. The schematic depicted in Example 2A represents that also used to measure LTP in this example.

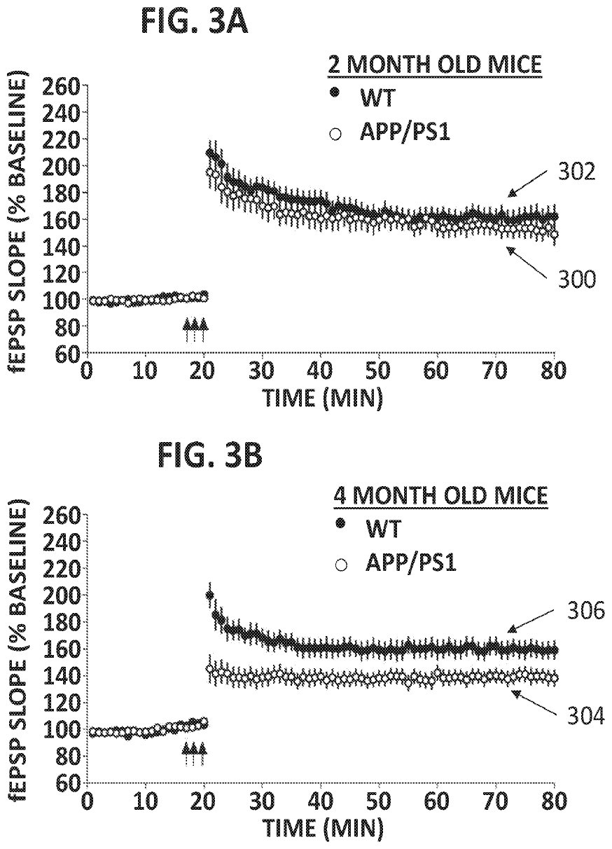

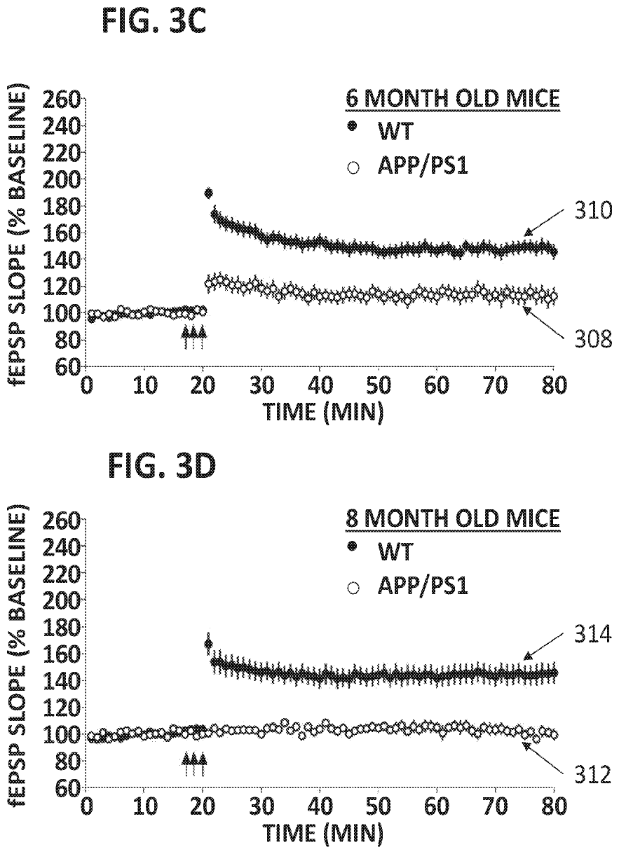

A) FIG. 3A: LTP of synaptic responses in the CA1 region, measured as an increase in the magnitude of CA1 field potentials following brief tetanic stimulation of the Shaffer collateral input, was slightly lower in slices from 2-mos-old APP/PS1 mice (graph 300) compared to slices taken from age-matched WT mice (graph 302).

B) FIG. 3B: LTP of synaptic responses in the CA1 region was further lowered in slices from 4-mos-old APP/PS1 mice (graph 304) compared to slices taken from age-matched WT mice (graph 306).

C) FIG. 3C: LTP of synaptic responses in the CA1 region was dramatically lowered in slices from 6-mos-old APP/PS1 mice (graph 308) compared to slices taken from age-matched WT mice (graph 310).

D) FIG. 3D: LTP of synaptic responses in the CA1 region was absent in slices from 8-mos-old APP/PS1 mice (graph 312) compared to slices taken from age-matched WT mice (graph 314).

### Example 4

### We Wuantified Acute Recruitment of CD11c-Labeled Dendritic Cells into Brain of Tg4510 Tauopathy Mice

The data in Example 4 indicate that recruitment of CD11c+ cells into brain is a pathology elicited by mutations known to cause fronto-temporal dementia in humans and lead to neurofibrillary tangle formation. These data indicate there are common pathways underlying dendritic cell recruitment at different stages of AD and that there is a wide treatment window for therapies that block dendritic cell recruitment.

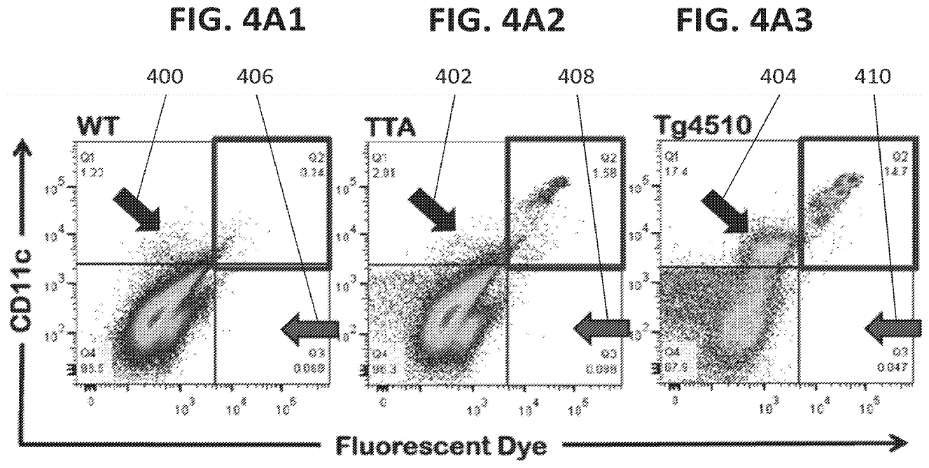

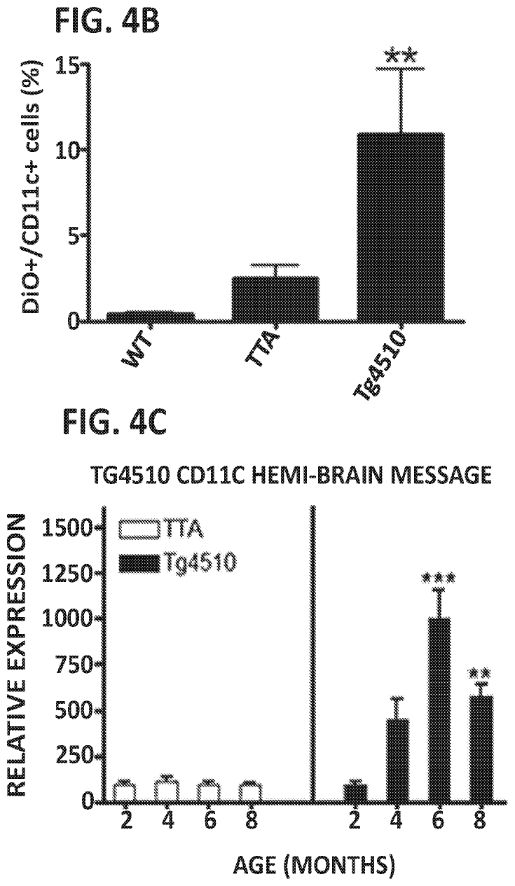

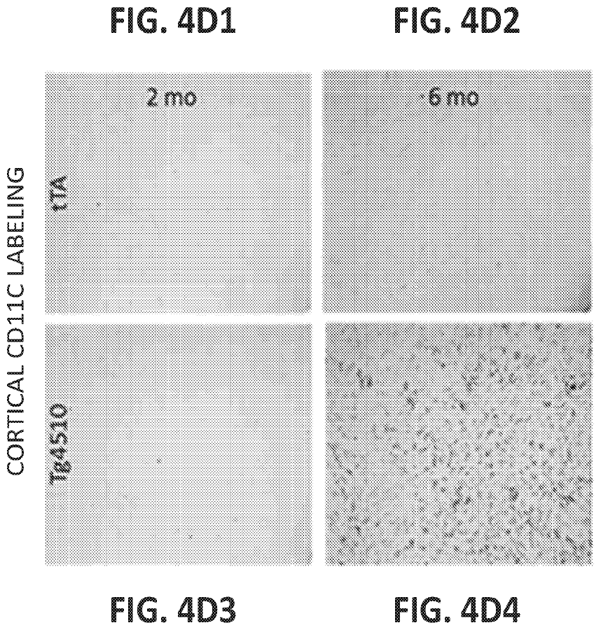

A) FIG. 4A1-4A3: “WT” includes the result from 6 month old wild-type mice that were litter mates to 6 month old Tg4510 transgenic mice, “TTA” includes the result from 6 month old mice that carried a tetracycline-controlled transcriptional activator gene used to regulate mutant tau, but in these mice no mutant tau is expressed. “TTA” were also litter mates to 6 month old Tg4510 transgenic mice. “Tg4510” includes the result from Tg4510 transgenic mice carrying the tetracycline-controlled transcriptional activator gene but in this case actively regulating expression of mutant tau. By non-specifically labeling blood-borne cells with the fluorescent membrane tracer DiO and using a “pulse-chase” design, we discriminate pre-existing dendritic cell populations in the brain (i.e. present prior to “pulse-labeling”, upper left quadrant). Note that this dendritic cell population is much larger in the Tg4510 AD mouse model than in WT or tetracycline-controlled transcriptional activator (tTA) control litter mates. Dendritic cell marker+/DiO+ double-labeled cells (upper right quadrant) are dendritic cells that have infiltrated the brain during the “chase” period. Note that these cells are present in the Tg4510 mouse samples, greatly attenuated in the tTA control, and virtually absent from the WT control. In all conditions, we did not observe a population of DiO⁺ cells in brain that were NOT labeled with the dendritic cell marker, indicating that there were no other major non-dendritic cell populations of recruited cells. Flow cytometry showed a selective accumulation of CD11c-labeled cells in the brains of Tg4510 transgenic mice relative to the tetracycline-controlled transcriptional activator and wild-type control mice. These were cells present in brain prior to DiO injection (blue arrows labelled 400 in FIG. 4A1 and 402 in FIG. 4A2). Flow cytometry also revealed that Tg4510, tetracycline-controlled transcriptional activator, and wild-type control mice all lacked a population of DiO-positive cells in brain that did not also label with the dendritic cell marker CD11c, indicating that there were no other major non-dendritic cell populations of recruited cells during the DiO labeling period (green arrows labelled 406 in FIG. 4A1, 408 in FIG. 4A2, and 410 in FIG. 4A3).

B) FIG. 4B:The increase in DiO⁺/CD11c⁺ cells in samples from the Tg4510 mice relative to tTA and WT litter mate control mice is indicated.

C) FIG. 4C: The relative expression levels of CD11c mRNA (gene designation is “Itgax”) increase in the brain of Tg4510 mice over months of age—2, 4, 6, and 8 mos old mice were compared. No such increase in CD11c mRNA occurs in age-matched tetracycline-controlled transcriptional activator control mice.

D) FIG. 4D1-4D4: Cells labeled for the DC-enriched marker CD11c, measured by immunohistochemistry, selectively increase as a function of age and transgene in the brains of Tg4510 neurofibrillary tangle-forming mice. The increase in immunolabeling is paralleled by increases in CD11c message (C above). Significantly, CD11c immunolabeling is concentrated near neurofibrillary tangle pathology. CD11c immunolabeling was low to undetectable in age-matched tTA control mice.

### Example 5

### Recruited Dendritic Cells can also be Detected by Imaging of Near-Infrared Fluorescence in Brain After Peripheral Uptake of the Tracking Dye Indocyanine Green

By non-specifically labeling blood-borne cells with the near infrared fluorescent tracking dye indocyanine green (ICG)—which is endocytosed by myeloid cells including dendritic cells—we used a “pulse-chase” design to visualize a dendritic cell population recruited into the brain using a near infrared scanner 24-48 hrs. after peripheral labeling.

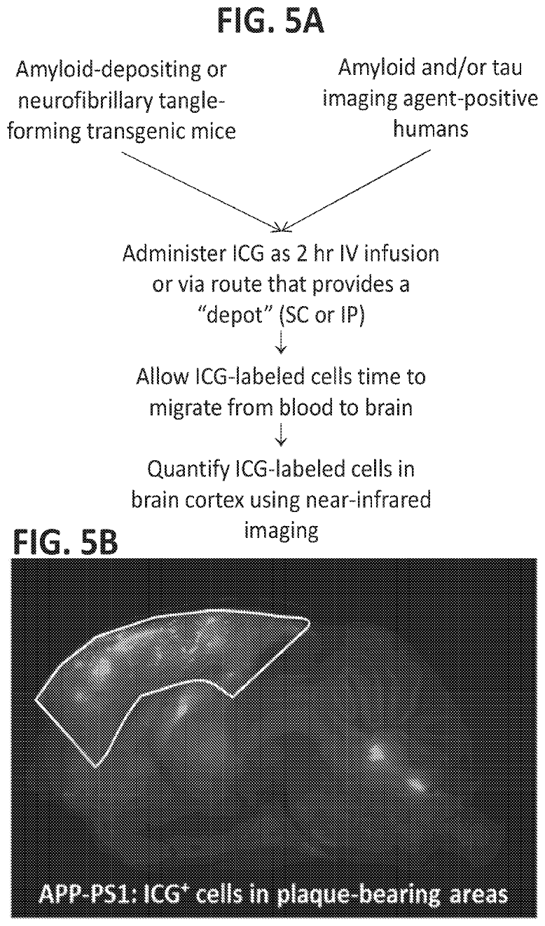

A) FIG. 5A: Schematized protocol for labeling peripheral innate immune cells in mice or humans, then measuring the recruitment of dendritic cells in brain using near infrared imaging of brain either ex vivo or in vivo.

B) FIG. 5B: Near infrared scan of the medial surface of a 12 mos old APP/PS1 mouse brain 48 hrs. post IP injection with 1 mg ICG. Recruitment of dendritic cells over the 48 hrs. “chase” period is primarily seen in areas of high amyloid plaque density, especially the cerebral cortex. In this figure, the cerebral cortex is outlined as a “region of interest”. Fluorescence signal intensity within such delineated anatomical regions can be quantified using software on the near infrared scanner.

### Example 6

### ICG-Labeled Cells are Specifically Recruited into Brain of Transgenic Vs. Wild-Type Mice, and Preferentially Accumulate in Areas of High Amyloid Plaque Pathology

In this example, 15 months old wild-type (WT) and APP/PS1 transgenic mice were injected IP with 1 mg ICG in a 500 ul injection volume, then sacrificed 2 days later. Mice were anesthetized and perfused with phosphate-buffered saline, brains were removed and hemisected sagitally. A hemibrain was then placed medial side down on a LiCor Odyssey infrared scanner and scanned in the 800 nm fluorescence channel.

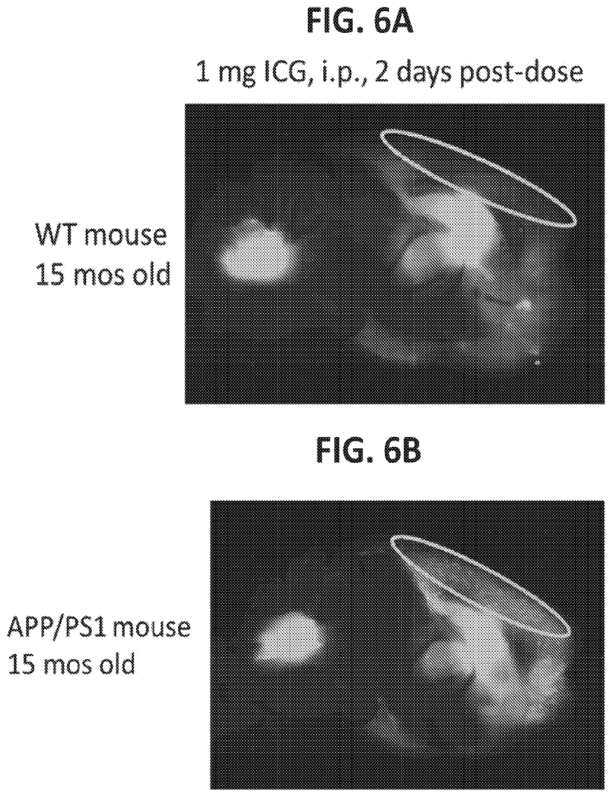

A) FIG. 6A: Infrared image of the medial surface of a 15 mos. old litter mate WT mouse 2 days post IP injection of 1 mg ICG. The oval highlights cerebral cortex.

B) FIG. 6B: Infrared image of the medial surface of a 15 mos old APP/PS1mouse 2 days post IP injection of 1 mg ICG. The oval highlights cerebral cortex, an amyloid plaque-rich region in APP/PS1 mice in which recruited dendritic cells accumulate.

### Example 7

### Pre-Injection of 3 mg/kg of the Ribosomal Toxin Saporin Conjugated to a CD11c mAb Followed by ICG Injection in APP/PS1 Transgenic Mice Abolishes Dendritic Cell Recruitment into Brain Over the Next 48 hrs., an Effect Mirrored by Loss of CD11c+ Cells Surrounding Amyloid Plaques

The CD11c mAb targets saporin to dendritic cells. Since saporin is only toxic when internalized, this treatment leads to selective ablation of CD11c⁺ dendritic cells in the periphery. Saporin-CD11c mAb is a 235 kDa protein that is unable to cross the blood-brain barrier and acts only in the periphery. Ablation of peripheral dendritic cells leads in turn to an absence of ICG-labeled cells in brain, corroborative evidence that the ICG signal originates from dendritic cells recruited from the blood. The loss of CD11c+ cells surrounding amyloid plaques corroborates this finding and indicates a rapid turnover of dendritic cells in the brain.

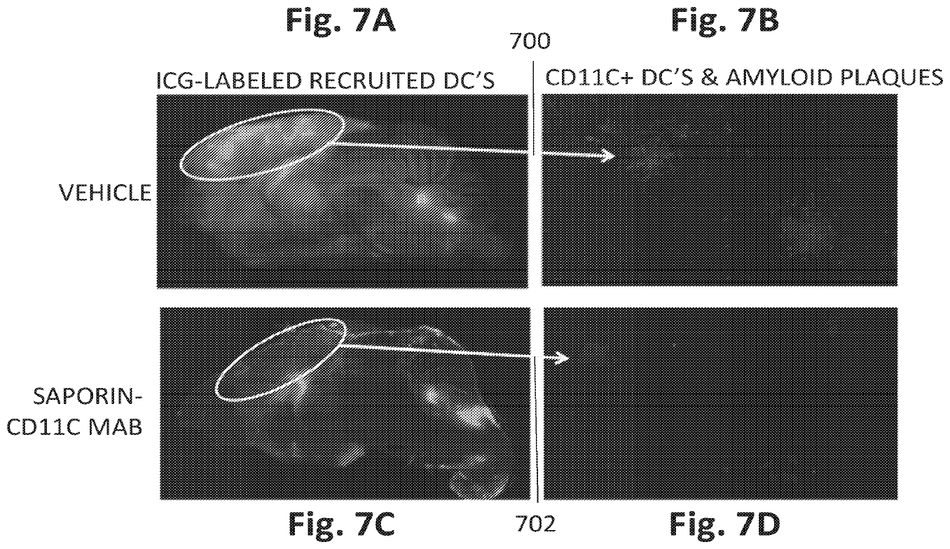

A) FIG. 7A: Infrared image of the medial surface of a 12 mos old APP/PS1 mouse brain. The mouse was injected with 200 μl phosphate-buffered saline IP, followed 18 hrs. later with an IP injection of 1 mg ICG in 500 μl distilled water. The mouse was anesthetized 2 d later and transcardially perfused with PBS. Brain was removed, hemisected, and fixed in 4% paraformaldehyde prior to imaging. The oval highlights cerebral cortex.

B) FIG. 7B: High magnification confocal immunofluorescent image of cerebral cortex taken from brain described above in FIG. 7A indicated by outlined region and arrow 700. CD11c immunofluorescence is shown in red (N418 clone primary) and amyloid is labeled in blue (AmyloGlo).

C) FIG. 7C: Infrared image of the medial surface of a 12 mos old APP/PS1 mouse brain. The mouse was injected with 3 mg/kg CD11c mAb (N418 clone) conjugated to saporin in 200 μl phosphate-buffered saline IP, followed 18 hrs. later with an IP injection of 1 mg ICG in 500 μl distilled water. The mouse was anesthetized 2 d later and transcardially perfused with PBS. Brain was removed, hemisected, and fixed in 4% paraformaldehyde prior to imaging. The oval highlights cerebral cortex.

D) FIG. 7D: High magnification confocal immunofluorescent image of cerebral cortex taken from brain described above in FIG. 7C indicated by outlined region and arrow 702. CD11c immunofluorescence is shown in red (N418 clone primary) and amyloid is labeled in blue (AmyloGlo).

Principal Methods

Key Reagents: Vibrant® DiO Cell labeling solution is purchased from Invitrogen. Fc Block (CD16/CD32 mAb cocktail), FITC- or APC-conjugated monoclonal antibodies (mAb) against mouse CD11b (clone M170, rat IgG2b), CD11c (hamster IgG1), MHCII (rat IgG2a), CD86 (rat IgG1), Ly6C (rat IgG2b), CD45 (rat, IgG2b), CD209 (rat IgG2a) and matching isotypes are purchased from BD Biosciences. Indocyanine green is purchased from Fisher Scientific.

Brain Immune Cell Infiltration Assay with DiO: Hemi and wild type litter mates are injected intravenously (i.v.) though the tail vain with 100 μL of 1 mM Vibrant® DiO Cell labeling solution in 1× PBS. A group of animals injected with vehicle (1× PBS) are used as negative controls for ex vivo flow cytometry studies. Two i.v. injection total are given 24 h apart. At the end of 48 h, mice are anesthetized with isofluorane and transcardially perfused with 1× HBSS (without CaCl, MgCl, MgSO₄) and Heparin (10 units/ml) at a flow rate of 3 mL/min for 7 min. Forebrains are collected in 5 mL 1× HBSS and stored in wet ice shielded from light until completion of tissue collection. Brains (cerebellum removed) are transferred to snap cap tubes, each containing 4 mL of digestion buffer containing warm DMEM Glutamax without sodium pyruvate (Invitrogen) and 60 U of papain (26.4 U of protein/mg; Worthington labs). Brains are incubated with the digestion buffer in a 37° C., 4% CO2 incubator for 2 h. Gentle trituration with a 10 mL pipette is performed every 30 min. Following digestion and trituration, 10 mL of 100% FBS is added to the brain homogenates to stop enzymatic digestion followed by filtration through 0.45 μm strainers (Thermofisher Scientific). The flow through is centrifuged at 500×g for 10 min at 25° C. Cell pellets from each brain are gently suspended in 15 mL warm 30% cell-culture grade endotoxin-low Percoll prepared from dilution of 100% isotonic Percoll solution pH 8.5-9 (Sigma) in Automax® buffer (Miltenyi). Samples are centrifuged at 500×g for 15 min at 25° C. without a break. Floating myelin is removed using 2 mL plastic Pasteur pipettes (Thermofisher Scientific) and by filtration through 0.45 μm strainers. Flow through is supplemented with 35 mL of Automax® buffer and centrifuged at 500×g for 15 min at 25° C. Fluid is decanted and pellet re-suspended in 1 mL Automacs® buffer for immunostaining and flow cytometry analysis.

Ex vivo Flow Cytometry: Cells suspended in Automacs® buffer are counted using a hemocytometer and incubated with Fc block for 5 min, washed once with cold Automacs® buffer, followed by centrifugation at 500×g for 2 min. Cell pellets are suspended in 100 μL fluorescence-conjugated mAbs (1 μg of mAb/106 cells) for 30 min at 4° C. and shielded from light. Post-incubation cells are washed thrice in Automacs® buffer followed by centrifugations at 500×g for 2 min at 4° C. Cells are then fixed with 2% paraformaldehyde and data are acquired on a BD FACSVerse™. Analyses are performed using FlowJo and GraphPad software.

Brain Immune Cell Infiltration Assay with Indocyanine Green (ICG): Hemi and wild type litter mates are given a single intraperitoneal (i.p.) injection with 500 μL of 2 mg/ml ICG in sterile water. Mice injected with the same volume of vehicle (sterile water) are used as negative controls for ex vivo infrared scanning of hemi-brains harvested from the mice. 48 h post-ICG injection, mice are anesthetized with isofluorane and transcardially perfused with 1× HBSS (without CaCl, MgCl, MgSO4) and Heparin (10 units/ml) at a flow rate of 3 ml/min for 7 min. Forebrains are collected in 5 mL 1× HBSS and stored in wet ice shielded from light until completion of tissue collection. Brains, including cerebellum and brainstem as control comparator regions are sagittally hemisected with a sharp razor blade. One hemibrain is transferred to 5 ml 1× HBSS for transfer to the infrared scanner, while the other hemibrain is fixed in 5 ml 4% paraformaldehyde in 1× HBSS for 2 hrs., then transferred to 30% sucrose in 1× HBSS to cryopreserve for cryostat sectioning. 15 ml conical tubes containing hemibrains are stored on ice and protected from light between procedures.

Image Analysis by Infrared Scanning: The hemibrain in 1× HBSS is transferred to the glass scanning plate of a LiCor Odyssey infrared imager, medial side down, and scanned at 0 mm focal offset above plate, 21 um resolution, high quality scan setting. ICG signal is quantified by defining “regions of interest” as detailed in the scanning software, then quantifying the infrared fluorescent signal in the 800 nm emission channel from matched area sizes across different brains. Cerebral cortex and hippocampus are the primary areas in which ICG-positive cells accumulate, preferentially associating with amyloid plaques.

Image Analysis of Brain Sections: In studies where image analysis of medial hemibrain indicates a signal difference across experimental manipulations, a more extensive quantification of these signal differences is carried out by cutting 30 um brain sections from the cryopreserved hemibrain on a cryostat, collecting every 5^{th} section, and scanning slides mounted with these serial sections on the LiCor Odyssey infrared imager either with no further processing or after co-labelling with a primary antibody against a desired antigen conjugated to an infrared fluorophore emitting a fluorescent in the 700 nm channel of the LiCor This is a method for examining co-labeling of ICG+ cells with dendritic cell markers using the LiCor Odyssey. Slides/sections treated with such conjugated antibodies are first treated with Fc block (see reagents) to prevent non-specific labeling of Fc domains by the labeling antibody.

Tissue Histology and Immunofluorescence: Standard methods well known to those skilled in the art are used. Individual antibody titers used to date have been optimized for best combined signal when used in double and triple immuno-labeling studies.

Physiology Studies: Synaptic transmission and LTP assessments were made on in situ hippocampal slices prepared from APP/PS1 mice and their WT litter mate mice. These methods are well known to those skilled in the art.

1.-54. (canceled)

55. A method of determining the presence of neurodegenerative disease in a human patient,comprising: measuring dendritic cell migration into the brain of the patient.

56. The method of claim 55, wherein the neurodegenerative disease is caused by neuroinflammation.

57. The method of claim 55, wherein the neurodegenerative disease is Alzheimer's disease.

58. The method of claim 55, wherein measuring dendritic cell migration into the brain includes measuring the number of dendritic cells in the brain of the patient.

59. The method of claim 55, wherein measuring dendritic cell migration into the brain includes measuring the amount of dendritic cell biomarker in the brain of the patient.

60. The method of claim 59, wherein the dendritic cell marker includes CD11c, Dectin-1, CD103, and MHC-II.

61. The method of claim 55, wherein measuring the number of dendritic cells in the brain of the patient includes dendritic cells recruited from blood of the human patient into the brain.

62. The method of claim 55, wherein dendritic cell migration into the brain of the patient is a biomarker of neurodegenerative disease.

63. The method of claim 55, wherein measuring dendritic cell migration into the brain of the human patient includes labeling peripheral blood mononuclear cells of the human patient using an IV infusion of indocyanine green and measuring the amount of indocyanine green uptake into the brain of the humanhunran patient using near infrared imaging.

64. The method of claim 63, wherein measuring the amount of indocyanine green uptake into the brain of the human patient using near infrared imaging is done over time.

65. A method of determining the presence of neurodegenerative disease in a human patient, comprising:
a. measuring dendritic cell migration into the brain of the patient as a first biomarker; and
b. measuring at least one additional biomarker for neurodegenerative disease.

66. The method of claim65, wherein the neurodegenerative disease is caused by neuroinflammation.

67. The method of claim 65, wherein the neurodegenerative disease is Alzheimer's disease.

68. The method of claim 65, wherein measuring dendritic cell migration into the brain includes measuring the number of dendritic cells in the brain of the patient.

69. The method of claim 65, wherein measuring dendritic cell migration into the brain includes measuring the amount of dendritic cell biomarker in the brain of the patient.

70. The method of claim69, wherein the dendritic cell a er includes CU11c, Dectin-CD103, and MHC-II.

71. The method of claim 65, wherein measuring the number of dendritic cells in the brain of the patient includes dendritic cells recruited from blood of the human patient into the brain.

72. The method of claim 65, wherein the at least one additional biomarker is an amyloid imaging agent, a tau imaging agent or a TSPO imaging agent.

73. The method of claim 65, wherein measuring dendritic cell migration into the brain of the human patient includes labeling peripheral blood mononuclear cells of the human patient using an IV infusion of indocyanine green and measuring over time the amount of indocyanine green uptake into the brain of the human patient using near infrared imaging.

74. The method of claim 73, wherein measuring the amount of indocyanine green uptake into the brain of the human patient using near infrared imaging is done over time.
